(19) United States
(12) Patent Application Publication
(10) Pub. No.: US 2025/0082390 A1
(43) Pub. Date: Mar. 13, 2025

(54) HIGH-INTEGRITY R-WAVE TRIGGERED TUMOR THERAPY APPARATUS

(71) Applicant: Nanjing Zengcheng Biomedical Technology Co., Ltd., Nanjing, Jiangsu (CN)

(72) Inventors: Zhongyi LIU, Nanjing (CN); Jianhui GAO, Nanjing (CN); Yu CHEN, Nanjing (CN); Shuhang XU, Nanjing (CN); Zhijun FANG, Nanjing (CN); Chao LIU, Nanjing (CN); Jianwei XU, Nanjing (CN); Xinwei LUO, Nanjing (CN); Lei HE, Nanjing (CN); Ke ZHANG, Nanjing (CN); Wenjie LIU, Nanjing (CN); Chun ZHANG, Nanjing (CN); Pei AN, Nanjing (CN)

(73) Assignee: Nanjing Zengcheng Biomedical Technology Co., Ltd., Nanjing, Jiangsu (CN)

(21) Appl. No.: 18/398,371

(22) Filed: Dec. 28, 2023

(30) Foreign Application Priority Data
Sep. 12, 2023 (CN) 202311169888.7

(51) Int. Cl. A61B 18/12; A61B 5/33; A61B 5/352; A61B 34/00

(52) U.S. Cl. CPC A61B 18/1206; A61B 5/33; A61B 5/352; A61B 34/25; A61B 2017/0019; A61B 2018/00577; A61B 2018/00779; A61B 2018/00839; A61N 1/36002

(57) ABSTRACT

The present invention discloses a highly integrated R-wave triggered tumor therapy apparatus, and relates to the technical field of medical devices for ablation of tumors. The apparatus includes: a human body R-wave detection module for detecting a human body R-wave signal; a signal generator for obtaining a narrow pulse signal with nanometer width according to the human body R-wave signal; a primary matching circuit; and a high-frequency transformer; where the primary matching circuit is used for matching the narrow pulse signal with nanometer width to an input side of the high-frequency transformer, so that the high-frequency transformer outputs a high-voltage signal needed for treatment of human bodies.

### CROSS REFERENCE TO RELATED APPLICATIONS

This application claims the benefit of priority to Chinese Patent Application No. 202311169888.7, filed Sep. 12, 2023, the contents of which are incorporated by referenced in their entirety.

### TECHNICAL FIELD

The present invention relates to the technical field of medical devices for ablation of tumors, and specifically, to a highly integrated R-wave triggered tumor therapy apparatus.

### BACKGROUND

As a new tumor ablation technology, nanoknife releases high-voltage pulses to form nanometer permanent perforations on tumor cells, which disrupts intracellular equilibrium and causes rapid cell apoptosis. Therefore, the nanoknife is also known as irreversible electroporation in medical practice. As compared to conventional thermal ablation of tumors including microwave ablation and radio frequency ablation, nanoknife tumor ablation is advantaged in retaining a normal vascular structure in an ablation area, high safety, activating a body immune system, and facilitating recovery of organ functions.

The nanoknife is mainly used in narrow pulses. High-voltage direct-current technologies and the minimum pulse width determine its minimum working duration and are the key features to distinguish the nanoknife tumor ablation from the microwave ablation and the radio frequency ablation. Because the nanoknife is used in relatively narrow pulses, it can implement perforation of pathological cells through electric field rather than conventional thermal ablation. In other words, the narrow pulse width indicates the good effect of the electric field on perforation. Due to the short duration of the narrow pulse, it is necessary to increase voltage to ensure the energy of a single pulse. Generally, the required voltage of the nanoknife is 10 KV to 30 KV.

At present, the solution used for available products on the market is that a power supply is charged by a high-voltage capacitor when out of service, or instantaneously discharged in service. This puts forward extremely high requirements for the design of the power supply of the nanoknife. At the same time, due to the charging and discharging characteristics of the high-voltage capacitor, if the relatively narrow high-voltage pulse needs to be output, the volume of the high-voltage capacitor should be extremely large, and therefore the volume of the product will also be large without doubt. Due to the limitations of the high-voltage capacitor, the narrowest pulse width of the existing ablation apparatus on the market can only be a few micro-seconds and difficult to reach the nanometer level, and its transient voltage is difficult to reach tens of kilovolts. This causes the undesired therapeutic effect.

In addition, when the nanoknife irritates cancer cells in the body, blood pressure and heart rate rise for patients, so irritation exerted by the nanoknife needs to align with the heart rate. At present, the existing ablation apparatuses on the market do not have the R-wave detection function, an external electrocardiogram synchronizer is needed to output electrocardiogram R-wave signals to the nanoknife for signal synchronization. This is disadvantageous to the miniaturization of the product.

### SUMMARY

The present invention provides a highly integrated R-wave triggered tumor therapy apparatus, which can alleviate the above problems.

In order to alleviate the above problems, a technical solution adopted in the present invention is as follows:
a highly integrated R-wave triggered tumor therapy apparatus is provided and includes:
a human body R-wave detection module for detecting a human body R-wave signal;
a signal generator for obtaining a narrow pulse signal with nanometer width according to the human body R-wave signal;
a primary matching circuit; and a high-frequency transformer; where the primary matching circuit is used for matching the narrow pulse signal with nanometer width to an input side of the high-frequency transformer, so that the high-frequency transformer outputs a high-voltage signal needed for treatment of human bodies.

In a preferred embodiment of the present invention, the highly integrated R-wave triggered tumor therapy apparatus further includes a power detection protection module, where the power detection protection module is used for detecting signal power at an output side of the high-frequency transformer and feeding the signal power back to the signal generator, to correct the narrow pulse signal with nanometer width output by the signal generator and ensure the constant output power of the high-frequency transformer.

In a preferred embodiment of the present invention, the highly integrated R-wave triggered tumor therapy apparatus further includes a man-computer interaction platform, where the man-computer interaction platform includes a touch 7-inch display screen based on a quad-core Cortex-A53 processor and an android10 operating system, and the display screen is used for configuring signal pulses and voltage parameters, and displaying the treatment conditions of the human bodies.

In a preferred embodiment of the present invention, when the detected signal power at the output side of the high-frequency transformer is greater than a protection value, the signal generator stops outputting the narrow pulse signal.

In a preferred embodiment of the present invention, the signal generator includes an FPGA chip, a DA conversion chip, a primary operational amplifier and a secondary operational amplifier that are sequentially connected in series from the input side thereof to the output side thereof; and the FPGA chip is electrically connected with the human body R-wave detection module and the power detection protection module.

In a preferred embodiment of the present invention, the FPGA chip is used for generating a 14 bit digital narrow pulse signal according to the human body R-wave signal; the DA conversion chip is used for converting the 14 bit digital narrow pulse signal into a 3.3V analog narrow pulse signal; the primary operational amplifier is used for converting a single-ended signal to a differential signal based on the 3.3V analog narrow pulse signal to obtain a ±3.3V analog narrow pulse signal; and the secondary operational amplifier is used for amplifying differential voltage on the ±3.3V analog narrow pulse signal to obtain a ±12V analog narrow pulse signal as the narrow pulse signal with nanometer width finally output by the signal generator.

In a preferred embodiment of the present invention, a method for generating a 14 bit digital narrow pulse signal includes the following steps:
S1. acquiring, by the FPGA chip, a signal generation control instruction;
S2. judging whether the signal power at the output side of the high-frequency transformer is greater than a protection value; if the signal power is greater than the protection value, stopping generating the 14 bit digital narrow pulse signal by the FPGA chip; or if the signal power is not greater than the protection value, proceeding Step 3;
S3. judging whether the signal power at the output side of the high-frequency transformer is higher than required power; if the signal power is higher than the required power, generating a power adjustment parameter to reduce driving of the DA conversion chip, and then proceeding Step 4; or if the signal power is not higher than the required power, generating a power adjustment parameter to increase driving of the DA conversion chip, and then proceeding Step 4; and
S4. generating an initial pulse signal according to the signal generation control instruction; and obtaining the 14 bit digital narrow pulse signal through signal delay output according to the initial pulse signal, the power adjustment parameter and the human body R-wave signal.

Compared with the prior art, the present invention has the beneficial effects that:
1) A high-voltage pulse is not generated by a conventional capacitor discharge way, but signals are amplified through the high-frequency transformer; subsequently, in a case that output transient voltage is ensured to reach tens of kilovolts, the signal generator capable to output the pulse signal with nanometer width and the small-volume high-frequency transformer can be adopted, so that the volume of the tumor therapy apparatus is reduced;
2) The pulse width can be really several nanometers. A narrow therapeutic pulse reduces the energy of a single pulse, thereby reducing damage to benign tissues around pathological cells;
3) The tumor therapy apparatus is integrated with an electrocardiogram R-wave detection module, and an external electrocardiogram synchronizer is not needed, so that the volume of the tumor therapy apparatus is reduced; and
4) The closed-loop adjustment of power is realized from a signal source according to a feature that DA of the signal generator can adjust both pulse width and signal amplitude, and real-time detection of back-end power.

To make the foregoing objects, features and advantages of the present invention more comprehensible, the following gives detailed descriptions for embodiments of the present invention with reference to accompanying drawings.

### BRIEF DESCRIPTION OF THE DRAWINGS

To describe the technical solutions in embodiments of the present invention more clearly, the following briefly describes the accompanying drawings required for describing the embodiments. It is appreciated that the accompanying drawings below only show some embodiments of the present invention and thus should not be considered as limitations on the scope. A person of ordinary skill in the art may still derive other related drawings from the accompanying drawings without creative efforts.

FIG. 1 shows a system block diagram of a highly integrated R-wave triggered tumor therapy apparatus according to the present invention;

FIG. 2 shows a flowchart of generation of an FPGA digital narrow pulse signal according to the present invention;

FIG. 3 shows a flowchart of processing a narrow pulse signal by an analog circuit of a signal generator according to the present invention; and

FIG. 4 shows a flowchart of extraction of a human body R-wave signal according to the present invention.

### DESCRIPTION OF EMBODIMENTS

To make the objectives, technical solutions, and advantages of the embodiments of the present invention clearer, the following clearly and completely describes the technical solutions in the embodiments of the present invention with reference to the accompanying drawings in the embodiments of the present invention. Apparently, the described embodiments are some but not all embodiments of this application.

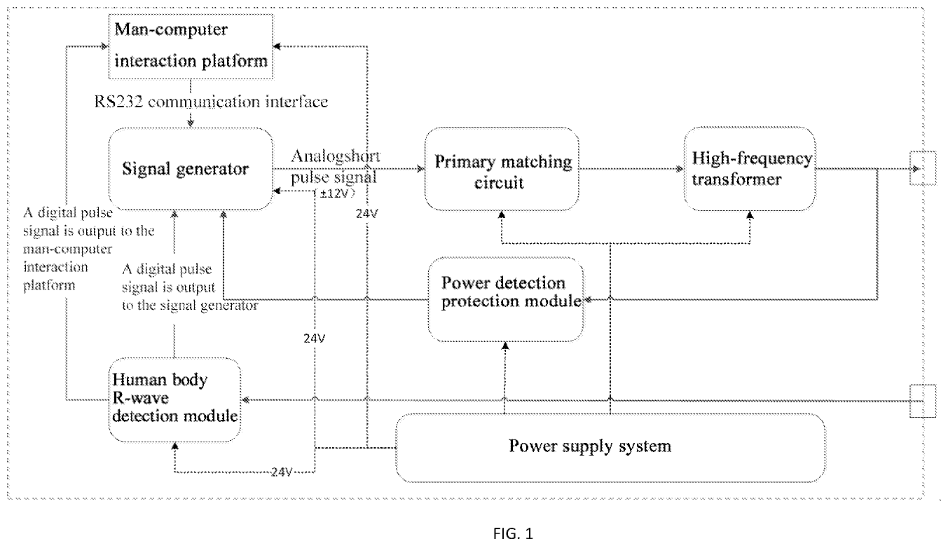

Referring to FIG. 1, the highly integrated R-wave triggered tumor therapy apparatus of the present invention includes a human body R-wave detection module, a power detection protection module, a man-computer interaction platform, a signal generator, a primary matching circuit, a high-frequency transformer, and a power supply system, where the signal generator, the primary matching circuit, the high-frequency transformer, and the power supply system are sequentially connected in series.
1. The man-computer interaction platform is used for configuring basic parameters of the present invention and displaying the treatment condition, including setting of signal pulse and voltage. An outsourced touch 7-inch display screen based on a quad-core Cortex-A53 processor and an android10 operating system is used in the man-computer interaction platform.

The man-computer interaction platform of the present invention has the following functions:
1) Configuration of basic parameter, treatment switching and other inputs, as well as R-wave, treatment pulse timing display and other contents of the man-computer interaction platform;
2) During treatment, therapeutic parameters can be automatically calculated through the calculation unit of the man-computer interaction platform according to the input type, volume, size and other parameters of the tumor for reference by physicians; in addition, similar cases stored in a back-end server can also be used to provide therapeutic parameters of existing cases for reference;
3) Hardware is equipped with 5G network, Bluetooth and wired network, which meets the hardware requirements for telemedicine according to the present invention to facilitate remotely monitoring devices by a manufacturer, and meets the remote communication between an user and the manufacturer to facilitate directly implementing the remote guidance and assistance to the user by the manufacturer.
2. The signal generator is a signal source of a pulse signal required for treatment, and can receive an instruction signal sent by the man-computer interaction platform and output a narrow pulse signal with nanometer width required for treatment according to a human body R-wave signal.

The signal generator of the present invention is crucially important, and needs to complete the configuration of corresponding signal parameters, the generation of narrow pulse signals and the control of output pulse power according to configuration instructions of the man-computer interaction platform.

In the present invention, the signal generator is of a structure including FPGA+DA+operational amplifier and includes an FPGA chip, a DA conversion chip, a primary operational amplifier and a secondary operational amplifier that are sequentially connected in series from the input side thereof to the output side thereof; and the FPGA chip is electrically connected with the human body R-wave detection module and the power detection protection module.

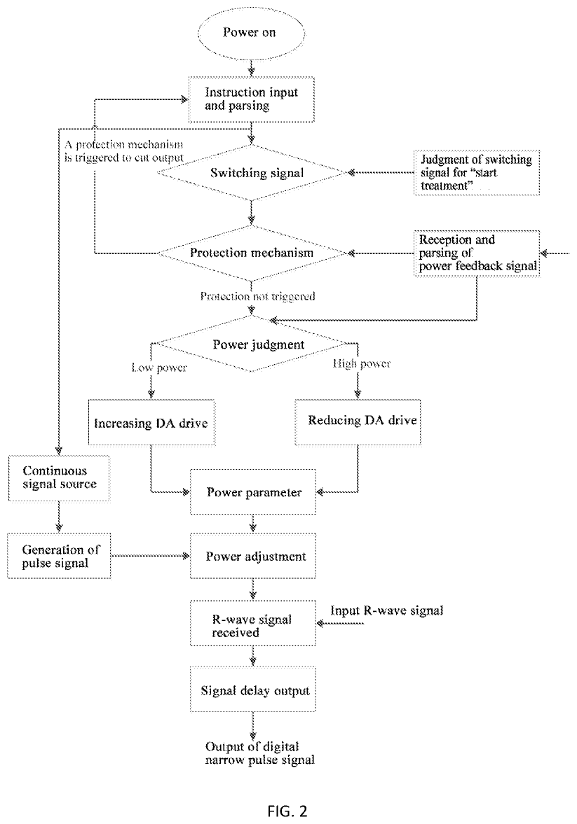

The FPGA chip is the control center of the signal generator and is mainly to communicate with the man-computer interaction platform, including reception of the instructions, uploading of a status instruction, reception of the human body R-wave signal, reception of a power detection signal (signal power at the output side of the high-frequency transformer) and generation of a DA driving signal. The FPGA chip of the present invention is XC6SLX25 of xilinx, and a basic execution process thereof is as shown in FIG. 2.

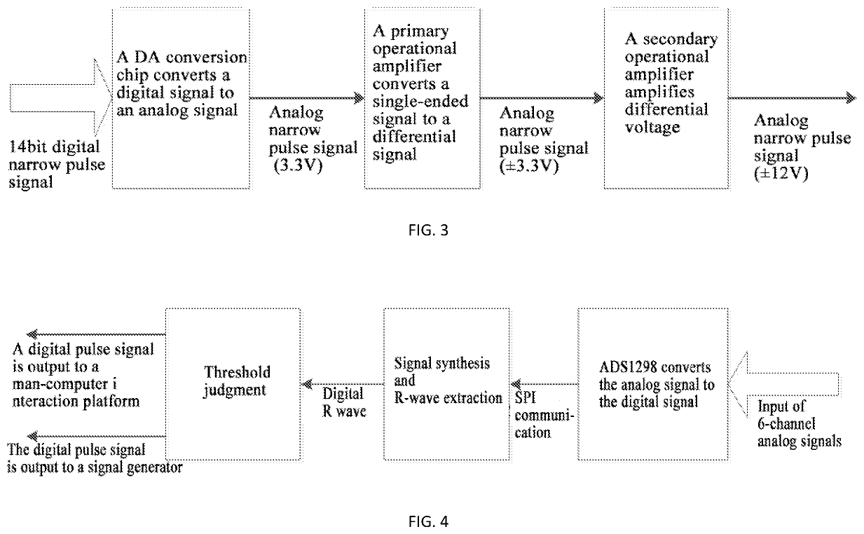

A digital signal output by the FPGA chip can be converted into an analog signal through the DA conversion chip; and the DA conversion chip of the present invention is 14 bit width and can achieve output of a 42 dB dynamic range, with a maximum output voltage of 3.3V. In this case, the signal also needs to be amplified by the primary operational amplifier and the secondary operational amplifier before outputting a ±12V narrow pulse signal with voltage and current matching with those of a next operational amplifier, and the ±12V narrow pulse signal is a narrow pulse signal with nanometer width required for treatment of the human body, as shown in FIG. 3.
3. The primary matching circuit is used to match the output signal of the signal generator with the input signal of the high-frequency transformer mainly for current amplification. The ±12V narrow pulse signal output by the signal generator is further subjected to current amplification, and the signal obtained through the primary matching circuit is a narrow pulse signal with high current and low voltage. In this case, the signal energy is already high enough, and then the signal can be amplified to a voltage value for treatment through a last-stage high frequency transformer.
4. The human body R-wave detection module is to align the narrow pulse signal and the heart rate during treatment; as shown in FIG. 4, external 6-channel detection inputs are used in the present invention; the external interfaces of the human body R-wave detection module are the same as those of common electrocardiographs; the FPGA with more constant time delay is mainly controlled when processing is performed at the back end thereof, thereby ensuring the constant delay between the actual signal of the front end and the sampling signal thereof; and the measured delay is a few microseconds and can fully meet the delay required for treatment. As the human body R-wave signal is a weak signal and generally only a few mV or so, the AD conversion chip with high sensitivity and 24 bit resolution is used in the front end of the human body R-wave detection module, thereby ensuring the detection of the weak signal. In addition, to reduce the interference of the energy radiation of the high-frequency transformer on the human body R-wave detection module, the outside of a human body R-wave detection module circuit is shielded by a metal shielding enclosure, while the input power supply is an isolated power supply.
5. The power detection protection module is used for detecting signal power at an output side of the high-frequency transformer and feeding the signal power back to the signal generator, to correct the narrow pulse signal with nanometer width output by the signal generator and ensure the constant output power of the high-frequency transformer, as shown in FIG. 2. When the detected signal power is greater than a protection value, a protection mechanism is triggered and output of the signal generator is closed.

The foregoing descriptions are merely preferred embodiments of the present invention and are not intended to limit the present invention. Persons skilled in the art understand that the present invention may have various modifications and variations. Any modification, equivalent replacement, or improvement made within the spirit and principle of the present invention shall fall within the protection scope of the present invention.

1. A highly integrated R-wave triggered tumor therapy apparatus, comprising:
a human body R-wave detection module for detecting a human body R-wave signal;
a signal generator for obtaining a narrow pulse signal with nanometer width according to the human body R-wave signal;
a primary matching circuit; and a high-frequency transformer; wherein the primary matching circuit is used for matching the narrow pulse signal with nanometer width to an input side of the high-frequency transformer, so that the high-frequency transformer outputs a high-voltage signal needed for treatment of human bodies.

2. The highly integrated R-wave triggered tumor therapy apparatus according to claim 1, further comprising a power detection protection module, wherein the power detection protection module is used for detecting signal power at an output side of the high-frequency transformer and feeding the signal power back to the signal generator, to correct the narrow pulse signal with nanometer width output by the signal generator and ensure the constant output power of the high-frequency transformer.

3. The highly integrated R-wave triggered tumor therapy apparatus according to claim 2, further comprising a man-computer interaction platform, wherein the man-computer interaction platform comprises a touch 7-inch display screen based on a quad-core Cortex-A53 processor and an android10 operating system, and the display screen is used for configuring signal pulses and voltage parameters, and displaying the treatment conditions of the human bodies.

4. The highly integrated R-wave triggered tumor therapy apparatus according to claim 2, wherein when the detected signal power at the output side of the high-frequency transformer is greater than a protection value, the signal generator stops outputting the narrow pulse signal.

5. The highly integrated R-wave triggered tumor therapy apparatus according to claim 2, wherein the signal generator comprises an FPGA chip, a DA conversion chip, a primary operational amplifier and a secondary operational amplifier that are sequentially connected in series from the input side thereof to the output side thereof; and the FPGA chip is electrically connected with the human body R-wave detection module and the power detection protection module.

6. The highly integrated R-wave triggered tumor therapy apparatus according to claim 5, wherein the FPGA chip is used for generating a 14 bit digital narrow pulse signal according to the human body R-wave signal; the DA conversion chip is used for converting the 14 bit digital narrow pulse signal into a 3.3V analog narrow pulse signal; the primary operational amplifier is used for converting a single-ended signal to a differential signal based on the 3.3V analog narrow pulse signal to obtain a ±3.3V analog narrow pulse signal; and the secondary operational amplifier is used for amplifying differential voltage on the ±3.3V analog narrow pulse signal to obtain a ±12V analog narrow pulse signal as the narrow pulse signal with nanometer width finally output by the signal generator.

7. The highly integrated R-wave triggered tumor therapy apparatus according to claim 6, wherein a method for generating a 14 bit digital narrow pulse signal comprises the following steps:
S1. acquiring, by the FPGA chip, a signal generation control instruction;
S2. judging whether the signal power at the output side of the high-frequency transformer is greater than a protection value; if the signal power is greater than the protection value, stopping generating the 14 bit digital narrow pulse signal by the FPGA chip; or if the signal power is not greater than the protection value, proceeding Step 3;
S3. judging whether the signal power at the output side of the high-frequency transformer is higher than required power; if the signal power is higher than the required power, generating a power adjustment parameter to reduce driving of the DA conversion chip, and then proceeding Step 4; or if the signal power is not higher than the required power, generating a power adjustment parameter to increase driving of the DA conversion chip, and then proceeding Step 4; and
S4. generating an initial pulse signal according to the signal generation control instruction; and obtaining the 14 bit digital narrow pulse signal through signal delay output according to the initial pulse signal, the power adjustment parameter and the human body R-wave signal.
